(19) United States
(12) Plant Patent Application Publication
(10) Pub. No.: US 2003/0121080 P1
(43) Pub. Date: Jun. 26, 2003

(54) SHRUB ROSE PLANT NAMED 'AUSCRYSTAL'

(75) Inventor: David C.H. Austin, Albrighton (GB)

Correspondence Address: KLARQUIST SPARKMAN, LLP, One World Trade Center, Suite 1600, 121 S.W. Salmon Street, Portland, OR, 97204, US

(73) Assignee: David Austin Roses Ltd.

(21) Appl. No.: 10/029,619

(22) Filed: Dec. 20, 2001

(51) Int. Cl. A01H 5/00

(52) U.S. Cl. PLT/107

(57) ABSTRACT

Shrub rose plant having warm, pale pink flowers; medium to dark green foliage with bronzy tinge; srong, almost prickleless, arching growth; and very flat, rosette-shaped flowers.

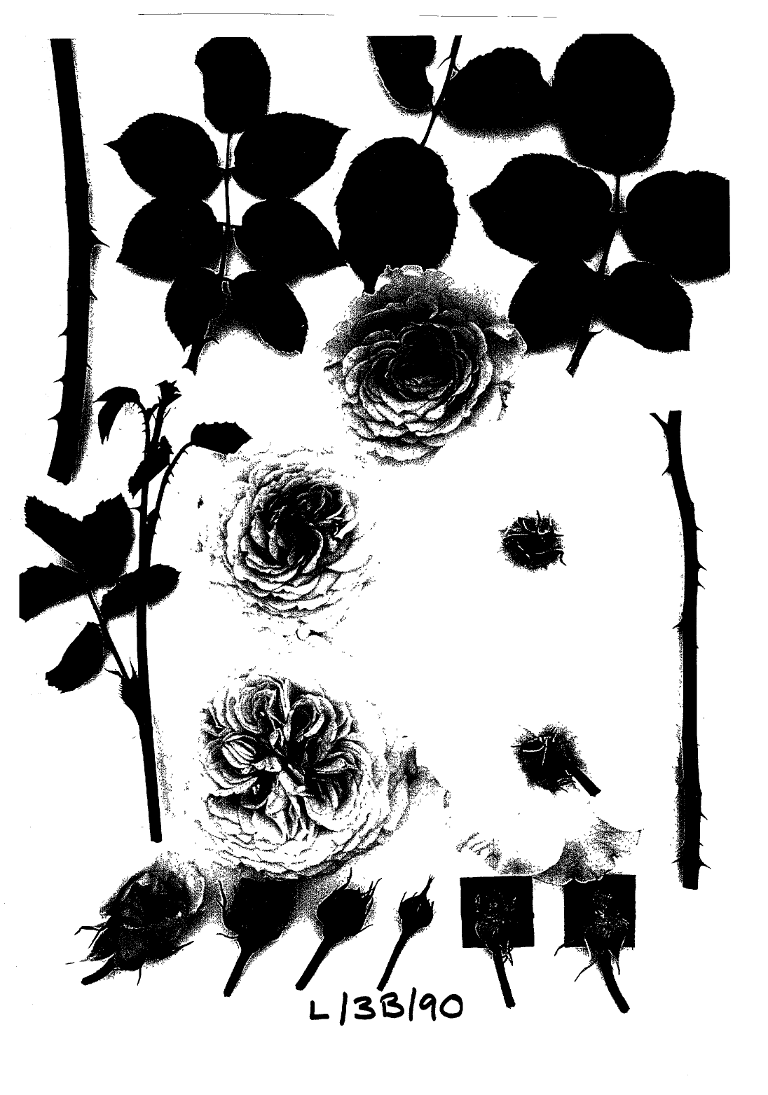

### SUMMARY OF THE INVENTION

The present invention relates to a new and distinct variety of rose plant of the shrub class which was originated by me by crossing the variety AUSblush, (unpatented), with an unnamed seedling (unpatented).

### BACKGROUND OF THE INVENTION

The primary objective of this breeding was to produce a new rose variety having a very free-flowering and healthy growth with flowers of the classic English rose shape and with a good fragrance.

### BRIEF SUMMARY OF THE INVENTION

The objective was substantially achieved, along with other desirable improvements, as evidenced by the following unique combination of characteristics that are outstanding in the new variety and that distinguish it from its parents, as well as from all other varieties of which I am aware:

1. Warm, pale pink flowers;

2. Medium to dark green foliage with bronzy tinge;

3. Strong, almost thornless, arching growth; and

4. Very flat rosette-shaped flowers.

Asexual reproduction of this new variety by budding on Rosa dumetorum laxa rootstock, as performed at Albrighton, England, shows that the foregoing and all other characteristics and distinctions come true to form and are established and transmitted through succeeding propagations.

### COMPARISON WITH PARENTS

AUScrystal plants have a strong, arching growth as compared to the more upright growth habit AUSBLUSH and its flowers of about 130 petals are flat as compared the cupped shaped flowers of AUSblush and the 50 petaled flower of the its pollen parent.

### COMPARISON WITH SIMILAR VARIETY

The most similar variety, AUSglisten, is more upright and branching and its foliage is smaller and rougher than that of AUScrystal.

### BRIEF DESCRIPTION OF THE ILLUSTRATION

The accompanying illustration shows typical specimens of the vegetative growth and flowers of this new variety in different stages of development, depicted in color as nearly true as it is reasonably possible to make the same in a color illustration of this character.

### DETAILED BOTANICAL DESCRIPTION

The following is a detailed description of my new rose cultivar with color descriptions using terminology in accordance with The Royal Horticultural Society (London) colour chart, except where ordinary dictionary significance of color is indicated.

Parentage:

Seed parent.—AUSblush (unpatented).

Pollen parent.—Unnamed Seedling (unpatented).

Classification:

Botanical.—Rosa hybrida.

Commercial.—Shrub.

### FLOWER

Observations made from plants of 8 years of age grown in a garden environment at Albrighton, England in July, 1998.

Blooming Habit: Recurrent.

Bud:

Size.—About 0.6 inches long when the petals start to unfurl.

Form.—The bud form is short, globular.

Color.—When sepals first divide, bud color is: Upper surface: Red Group 56B and 56C. Lower surface: Red Group 56C. When half blown: Upper surfaces of the petals are Red Group 56C; and the lower sides of the petals are Red Group 56C.

Sepals.—Color: Green Group 143C. Length: 0.8 inches. Width: 0.5 inches to 0.75 inches. Shape: Wide, goes to a point. Surface texture: Hairy edges. There are three normally appendaged sepals. There are two unappendaged sepals with hairy edges.

Receptacle.—Color: Green Group 143C with tint of Red-Purple Group 59A. Shape: Funnel. Size: Medium, about 0.2-0.3 inches×0.2-0.3 inches. Surface: Slightly hairy.

Peduncle.—Length: About 2 inches. Surface: Prickly. Color: Yellow-Green Group 144B with Red-Purple Group 59A blush on some. Strength: Stiff.

Blooms:

Size.—Medium. Average open size is 3 inches.

Borne.—In flat clusters of 3 to 7 blooms.

Stems.—Strength Normal. Average length is about 25-30 inches.

Form.—When first open bloom is: Flat. Permanence: Outer petals curl back.

Petalage.—Number of petals under normal conditions: 130.

Color.—The upper sides of the petals are Red Group 56C on upper portion and Red Group 56D in middle portion, and White Group 155B on the bottom portion. The major color on the upper side is Red Group 56A on inner petals and Red Group 55B on outer petal.

Discoloration.—The general tonality at the end of the first day: Red Group 55C. At the end of the third day: Red Group 55D. With aging the color changes to a bluing shade of pink.

Fragrance.—Strong. Character of Fragrance: Old Rose.

Petals:

Texture.—Average.

Shape.—Circulate and pointed at base (inner petals have a pointed shape at top of petal).

Margin.—Round with point.

Apex shape.—Rounded.

Base shape.—Pointed.

Form.—Tips slightly recurved on outer petals, edges slightly quilled on inner petals.

Arrangement.—Irregular. Petaloids in the center are few.

Persistence.—Petals hang on and dry.

Lastingness.—On the plant: Long. As a cut flower: Not tested.

Reproductive Parts:

Stamens.—Number: 64. Arrangement: Mixed with petaloids.

Anthers.—Length: 5/32 inch. Color: Yellow-Orange Group 14A.

Filaments.—Color: Yellow Group 4B. Length: ⅛ inch.

Pollen.—Color: Yellow-Orange Group 15A.

Pistils.—Number: 55. Styles: Color: Green-Yellow Group 1C. Stigmas: Color: Green-Yellow Group 1C.

Hips.—None formed.

### PLANT

Form.—Shrub, (English style).

Growth.—Vigorous, arching.

Mature plant.—Height: 4.5 feet. Width: 3.5 feet.

Rootstock.—Dr. Huey recommended for United States.

Foliage: Number of leaflets on normal mid-stem leaves: 5-7.

Size.—Large (5.5 inches long×3.75 inches wide).

Quantity.—Normal.

Color.—New foliage: Upper side: Yellow-Green Group 146A. Lower side: Yellow-Green Group 146C. Old foliage: Upper side: Green Group 139A. Lower side: Green Group 137A.

Leaflets:

Size.—About 2 inches long×2.25 inches wide.

Shape.—Broadly oval.

Tip shape.—Pointed.

Apex shape.—Rounded.

Texture.—Smooth.

Edge.—Serrated.

Serrations.—Small.

Petiole color.—Greyed-Red Group 178A.

Petiole rachis.—Color: Yellow-Green Group 144A and Greyed-Purple Group 183C blush. Underside: Smooth

Auricle shape, if present.—Long points.

Stipules.—Length: 0.75 inches, serrated.

Disease resistance.—Resistant to mildew, blackspot and rust under normal growing conditions.

Pest resistance.—Not tested.

Wood:

New wood.—Color: Greyed-Purple Group 183B and Yellow-Green Group 144B. Bark: Smooth.

Old wood.—Color: Yellow-Green Group 146B. Bark: Smooth.

Prickles:

Quantity.—On main canes from base: Ordinary. On laterals from main canes: Ordinary.

Form.—Hooked downward. Length: ⅓ inch.

Color when young.—Greyed-Purple Group 184A.

Color when mature.—Greyed-Purple Group 185B.

Small Prickles:

Quantity.—On main stalks: Few. On laterals: Few.

Color.—Violet Group 85B.

Climate Hardiness: Hardy in Zones 4-9.

I claim:

1. A new and distinct variety of rose plant of the shrub class, substantially as herein shown and described, characterized particularly by warm, pale pink flowers; medium to dark green foliage with bronzy tinge; strong, almost prickleless, arching branches; and very flat, rosette-shaped flowers.
